(19) United States
(12) Patent Application Publication
(10) Pub. No.: US 2008/0139923 A1
(43) Pub. Date: Jun. 12, 2008

(54) Method and apparatus for verifying occlusion of fallopian tubes

(75) Inventors: Mark F. Payne, Palo Alto, CA (US); James Stubbs, Palo Alto, CA (US); Moshe Zilversmit, Mountain View, CA (US)

Correspondence Address: CYTYC CORPORATION, 250 CAMPUS DRIVE, MARLBOROUGH, MA, 01752, US

(73) Assignee: Cytyc Corporation, Marlborough, MA (US)

(21) Appl. No.: 11/953,718

(22) Filed: Dec. 10, 2007

Related U.S. Application Data
(60) Provisional application No. 60/869,692, filed on Dec. 12, 2006.

(51) Int. Cl. A61B 5/055; A61B 8/14; A61B 5/053

(52) U.S. Cl. 600/411; 600/439; 600/547; 600/420

(57) ABSTRACT

A device for verifying occlusion of the fallopian tube in a female subject includes an elongate member dimensioned to be slidably disposed within the uterine cavity of the subject. An interrogation element is positioned at the distal end of the elongate member. The interrogation element may include a visualization element such as a camera, ultrasonic transducer, MRI imaging element, or the like. In still other aspects, the interrogation element may include an electrode that is used to measure the electrical properties of the occlusion site. For example, the electrode may be used to measure electrical impedance.

### FIELD OF THE INVENTION

The field of the invention generally relates to methods and devices used to verify or confirm occlusion of a body lumen. More specifically, the field of the Invention pertains to methods and devices for confirming or verifying fallopian tube occlusion.

### BACKGROUND OF THE INVENTION

Conventionally, bilateral tubal sterilization (BTS) has been used for sterilization in female patients. Typically, BTS is surgically accomplished by ligation of the fallopian tubes using one or more surgical approaches. More recently, various non-operative methods of achieving sterility have been developed as an alternative to conventional BTS procedures. For example, Conceptus, Inc. of San Carlos, Calif., has developed the ESSURE micro-insertion device which is deployed hysteroscopically. Also, Adiana, Inc. of Redwood City, Calif., has developed a hysteroscopically-placed device which uses low level radiofrequency energy to damage the fallopian tubes. A soft polymer matrix is left behind in the tube to facilitate closure. In both of these processes, sterilization is accomplished by occlusion of the intramural portion of the fallopian tubes.

These new, non-operative methods require some sort of post-procedure verification to ensure that the fallopian tube(s) have indeed been occluded. Typically, occlusion is verified after the sterilization procedure with the aid of hysterosalpinography (HSG). HSG is a radiographic technique in which a contrast media (e.g., oil or water soluble fluid containing a radiographically opaque compound of a material such as iodine) is injected slowly into the uterine cavity and fallopian tubes via a trans-cervically-placed cannula. Radiographic images are taken to delineate the inside of the uterus and fallopian tubes. Tubal occlusion is verified by the lack of contrast media past a specific location in the tube (or by lack of contrast media in certain anatomical spaces such as the pouch of Douglas). Unfortunately, HSG subjects the patient to ionizing radiation and the patient may potentially be sensitive to the contrast medium. Also, because HSG involves radiation, the procedure must be performed in a specialized suite or room suitable for radioactive procedures.

More recently, hysterosalpingo-contrast sonography (HyCoSy) has been developed for imaging the uterus and fallopian tubes. HyCoSy is an ultrasonic technique that is accomplished trans-vaginally after the uterus and fallopian tubes are filled with contrast media. Tubal occlusion (or lack thereof) is determined by the absence of contrast media past a specific location in the fallopian tube or by the absence of contrast media in other anatomical spaces (e.g., the pouch of Douglas). While HyCoSy does obviate the risks of radiation exposure, the method employs somewhat complex and expensive equipment. There is a need for a less complex device and method that can be used to verify and/or detect occlusions within the fallopian tube. Preferably the device and method should be able to verify occlusion in the intramural portion of the patient's fallopian tubes.

### SUMMARY

In one embodiment of the invention, a device for verifying occlusion of the fallopian tube in a female subject includes an elongate member dimensioned to be slidably disposed within the uterine cavity of the subject. An interrogation element is positioned at the distal end of the elongate member. The interrogation element may include a visualization element such as a camera, ultrasonic transducer, MRI imaging element, or the like. In still other aspects, the interrogation element may include an electrode that is used to measure the electrical properties of the occlusion site. For example, the electrode may be used to measure electrical impedance of tissue.

In another aspect of the invention, the device includes an elongate member having an ultrasonic transducer positioned at the distal end thereof. The ultrasonic transducer may be used to both emit and receive ultrasonic waves. The received ultrasonic signals may be translated into a visual representation of the occlusion site and displayed, for instance, on a monitor or display for viewing by the physician or other operator.

In another embodiment of the invention, the verification device is dimensioned such that it can be slidably disposed within a separate occlusion catheter or cannula. For example, the occlusion catheter may include an occlusion element such as a RF electrode at a distal end thereof. The occlusion catheter also includes a lumen through which the verification device is slidably disposed. In this regard, after the fallopian tube has been occluded, the verification device may be manipulated into place to view or interrogate the occlusion site to test for full occlusion.

The elongate member with the interrogation member may be made of any material suitable for accessing the occlusion site. The elongate member may be pre-formed or capable of being pre-formed into specific configuration (e.g., bent tip) for placement. Alternatively, the elongate member may be steerable by the user.

In another aspect of the invention, a device for verifying occlusion of the fallopian tube in a female subject includes an elongate member dimensioned to be slidably disposed within the uterine cavity of the subject. The elongate member includes an impedance-measuring electrode on a distal end thereof. During use, the impedance-measuring electrode may physically contact the occlusion site (or adjacent area) to measure the impedance in response to an applied electrical current. For example, impedance changes can detect and characterize fibrosis of the occlusion site in response to injury (e.g., applied RF energy for ablation of fallopian tube ostia). In one aspect, the impedance-measuring electrode is incorporated into an occlusion catheter or cannula. Alternatively, the verification device may be a separate device from the occlusion catheter or cannula.

In another aspect of the invention, a device for verifying the occlusion of the fallopian tube in a female subject includes an elongate member dimensioned to be slidably disposed within the uterine cavity of the subject. The device includes a dye applicator disposed on a distal end of the elongate member. A visualization tool may be disposed on a distal portion of the control member. Alternatively, a separate visualization tool like a hysteroscope may be used to interrogate the occlusion site for dye migration/penetration. The dye applicator may include a sponge, brush, or even a dye ejection port (or ports).

In another aspect of the invention, a method of verifying the occlusion of the fallopian tube in a female subject includes placing a dye-releasing agent within the fallopian tube at a location that is distal (toward the peritoneal space) of the putatative occlusion location. The fallopian tube may then be occluded by, for example, intentional damaging of the fallopian tube tissue (e.g., RF ablation of fallopian tube or ostial region), or inserting an occlusive device such as a coil or the like. The dye-releasing agent is preferably biodegradable and releases dye on the “back-side” of the occlusion after a preset amount of time. A visualization tool is then inserted into the uterine cavity and the occlusion site is inspected for the presence or absence of dye. The presence of dye indicates that full occlusion has not taken place.

### BRIEF DESCRIPTION OF THE DRAWINGS

The drawings illustrate the design and utility of various embodiments of the present invention, in which similar elements are referred to by common reference numerals. In order to better appreciate how the above-recited and other advantages and objects of the present inventions are obtained, a more particular description of the present inventions briefly described above will be rendered by reference to specific embodiments thereof, which are illustrated in the accompanying drawings. Understanding that these drawings depict only typical embodiments of the invention and are not therefore to be considered limiting of its scope, the invention will be described and explained with additional specificity and detail through the use of the accompanying drawings in which:

FIG. 1 is a partial cross-sectional view of the female reproductive system showing placement of an occlusion verification device according to one embodiment.

FIG. 2 is a partial cross-sectional view of the female reproductive system showing placement of an occlusion verification device according to another embodiment.

FIG. 3 is a partial cross-sectional view of the female reproductive system showing placement of an occlusion verification device according to another embodiment.

FIG. 4 is a partial cross-sectional view of the female reproductive system showing placement of an occlusion verification device according to another embodiment.

FIG. 5 is a schematic representation of a handheld device for verifying occlusion of a fallopian tube according to one aspect of the invention.

### DETAILED DESCRIPTION

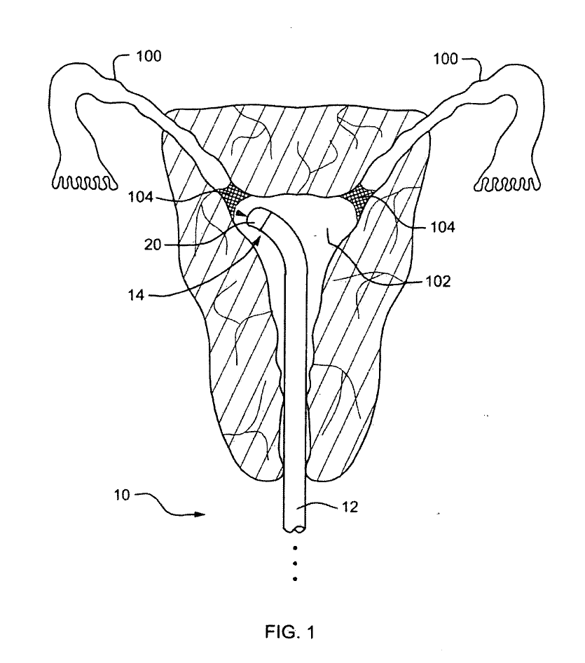

FIG. 1 illustrates an apparatus 10 for verifying whether or not a fallopian tube 100 of a female subject is occluded. Initially, it should be understood that the apparatus 10 is contemplated to be used after the fallopian tube 100 or tubes have been occluded. The apparatus 10 described herein may be used in conjunction with virtually any method used to occlude the fallopian tubes 100. For example, the fallopian tubes 100 may be occluded using a device such as a coil or insert. Alternatively, the fallopian tubes 100 may be occluded using a chemical method. In still another method of occlusion, the fallopian tubes may be intentionally damaged or scarred by the application of energy (e.g., RF energy).

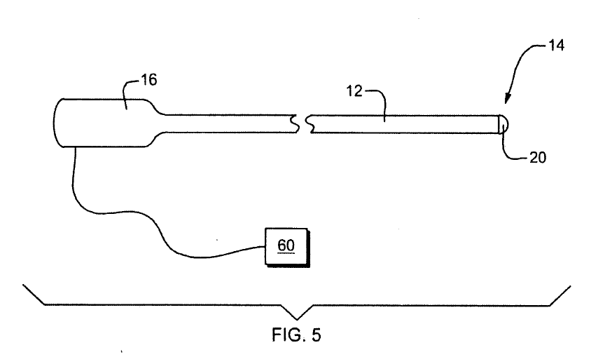

The apparatus 10 generally includes an elongate member 12 which may be formed as a cannula or catheter. In certain embodiments, the elongate member 12 may be formed as a handheld device. The elongate member 12 may be formed from a rigid or semi-rigid material such that the distal end 14 can be positioned in close proximity to the occluded fallopian tube 100. In one aspect, the elongate member 12 may be steerable by the user much in the manner that certain catheters can be manipulated or steered into position. For example, using a proximal hub or handle 16 (best seen in FIG. 5), the distal end 14 can steered into position to permit interrogation of the occluded fallopian tube 100. In another aspect of the invention, the distal end 14 of the elongate member 14 may be pre-formed or bent by the user prior to insertion into the uterine cavity 102 of the subject. For example, the distal end 14 of the device may be bent in the manner shown in FIG. 1 to permit better interrogation of the occlusion site 104.

Still referring to FIG. 1, in certain embodiments, the apparatus 10 includes an interrogation element 20 located on the distal end 14 of the elongate member 12. The interrogation element 20 may include an ultrasound transducer in some embodiments while in other embodiments, the interrogation element 20 may include a camera or the like that can visually inspect the occlusion site 104. With respect to the ultrasound transducer aspect, the interrogation element 20 may be used to visualize the occlusion site 104. An image may be displayed via a monitor (not shown) or the like that is connected to the device 10. Alternatively, the ultrasound-based interrogation element may be used to excite or vibrate the occlusive device inserted into the occlusion site 104. For example, after the occlusion process, the occlusion device (e.g., coil, insert, or the like) can be ultrasonically excited at a resonance frequency (or over a range of frequencies) to initiate vibrations. The nature and level of the excitation (e.g., vibration) can be used to evaluate the extent of fibrosis following the occlusion operation. Different vibrational characteristics can then be used to ascertain the level and extent of fibrosis. Incomplete fibrosis may indicate that a leak may be present in the fallopian tube 100. Along these same lines, the occlusion device may also be interrogated using an oscillating electromagnetic field in a similar manner depending on the nature and makeup of the occlusion device.

During or prior to use of the device 10, the uterine cavity 102 may be appropriately insufflated or distended with a gaseous or fluid medium. In this regard, insufflation or distension of the uterine cavity 102 may aid the inspection of the occlusion site(s) 104. For example, it may be easier to visualize or interrogate the ostial region of the fallopian tubes 100 using one or more of these methods.

In another embodiment of the invention, the interrogation element 20 may include an impedance measuring electrode. In this embodiment, an applied electrical current (A/C) is applied on or adjacent to the occlusion site 104 via the electrode. Backend circuitry 60 (e.g., as shown in FIG. 5) electrically coupled to the proximal end of the device 10 is then used to measure the impedance of the scar tissue. Depending on the extent and nature of fibrosis, the measured impedance changes. This change in impedance can be used to monitor tissue scaring or detect inadequate scarring which may be an indicator of leakage.

Still referring to FIG. 1, in another aspect of the invention, the interrogation element 20 may include magnetic resonance imaging (MRI) element. In this regard, the device 10 can operate as a handheld MRI-based device that can investigate and interrogate the occlusion site 104. In this embodiment, images of the occlusion site 104 may optionally be displayed on a monitor or the like for viewing. The physician or other healthcare provider can then inspect the patency of the fallopian tube 100 after the same has been occluded.

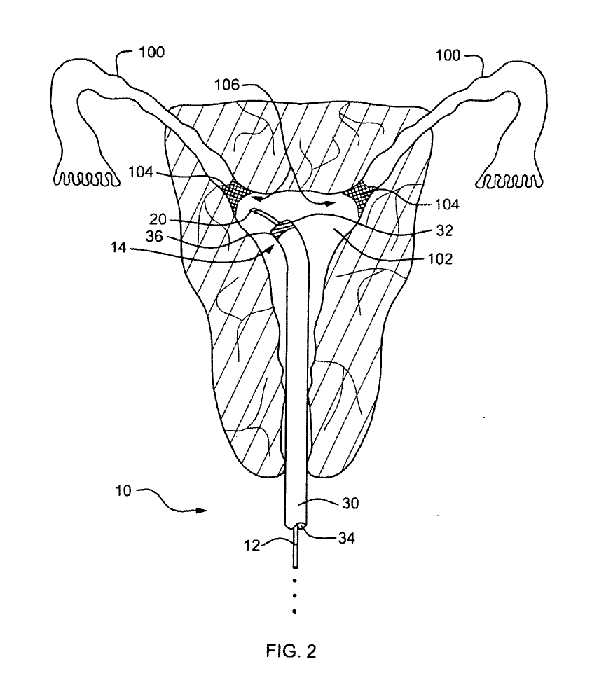

FIG. 2 illustrates an alternative embodiment of the device 10 in which the elongate member 12 and interrogation element 20 are sized to pass through a separate occlusion catheter or cannula 30. For example, as shown in FIG. 2, the occlusion catheter 30 may include a radiofrequency (RF) ablation electrode 32 located at the distal end 36 of the occlusion catheter 30. The RF ablation electrode 32 may be a monopolar or bipolar-based electrode. The RF ablation electrode 32 is electrically coupled to a RF generator (not shown) that is used to apply the RF energy. The RF ablation electrode 32 can be used, for example, to ablate tissue in and around the fallopian tube ostium 106 to cause a site of occlusion that prevents subsequent passage of sperm. In this regard, the RF ablation electrode 32 and associated occlusion catheter 30 are used to non-operatively sterilize the female subject.

In this embodiment, the occlusion catheter 30 is used to form the occlusion in the ostia of the fallopian tubes 100 through application of RF energy. After the occlusion operation, the device 10 can then be inserted distally into a lumen 34 of the occlusion catheter 30 to interrogate the occlusion site 104. Alternatively, the device 10 may already be pre-positioned or contained within the occlusion catheter 30. The device 10 can then be simply advanced distally to expose or otherwise position the interrogation element 20 in close proximity to the occlusion site 104. The occlusion catheter 30 and the device 10 may thus be used as two separate instruments or, alternatively, they may be combined into a unitary device or system.

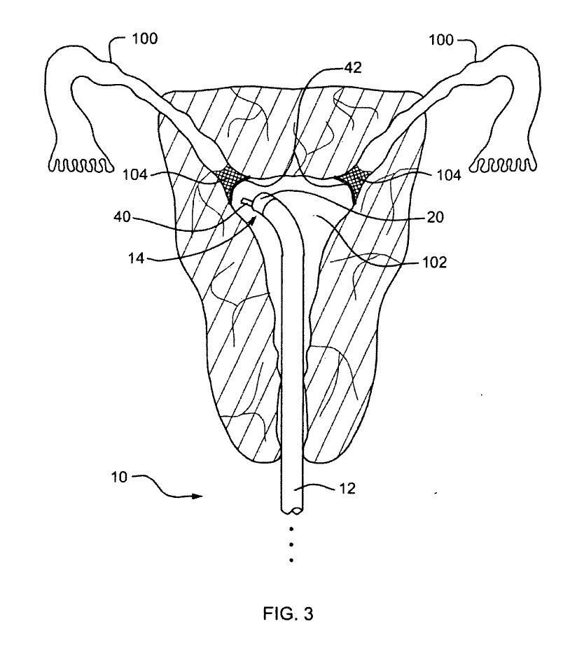

In another aspect of the invention, as shown in FIG. 3, the device 10 includes an elongate member 12 along with a interrogation element 20 positioned at the distal end 14. In this embodiment, the device 10 further includes a dye applicator 40 that is located at or near the distal end 14 of the elongate member 12. The dye applicator 40 is used to apply a thin film or coating 42 of dye onto the occlusion site 104. The dye applicator 40 may include a piece of absorbent material such as medical-grade sponge, a brush, or even a dye ejection port located on the distal end 14 of the elongate member 12. The elongate member 12 may contain a lumen or storage chamber inside (not shown) that is used to store and transport dye to the dye applicator 40. Application of the dye may be controlled through a proximally coupled syringe or the like (not shown) that can be depressed to aid in ejecting a thin film of dye over the occlusion site 104.

The dye, which may be a liquid, is allowed to migrate and penetrate into the occlusion site 104 and surrounding tissue. To aid this process, the uterine cavity 102 may be pressurized with an insufflation gas or even a fluid. Penetration of the dye through tissue may indicate the presence of a fissure or fistula through which the dye can “leak.” Penetration or leakage of the dye in the region adjacent to the occlusion site 104 is an indicator that tells the physician that total occlusion of the fallopian tube 100 has not been achieved. The interrogation element 20 may include a camera or other visualization tool that is used to assess the migration and penetration of the dye in and around the occlusion site 104.

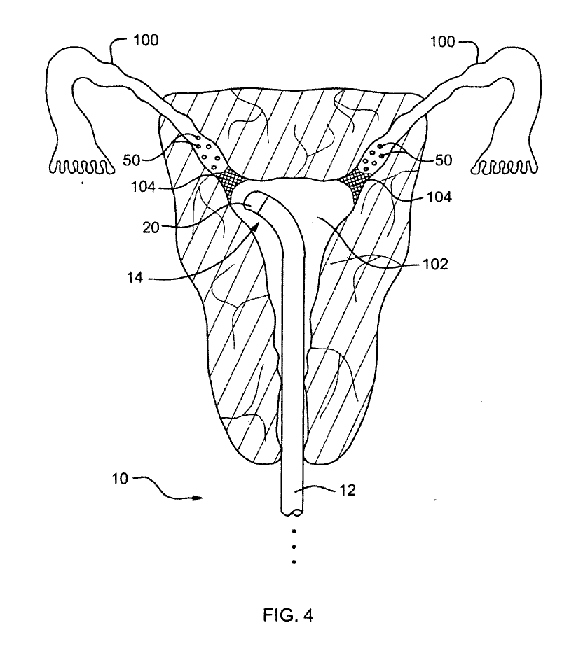

FIG. 4 illustrates yet another embodiment of the device 10. In this embodiment, prior to forming the occlusion within the fallopian tube 100, a dye-releasing agent 50 is placed within the fallopian tube 100. The dye-releasing agent 50 is placed distally with respect to the ostium 106 of the fallopian tubes 100. For example, the dye-releasing agent 50 may be deposited or ejected in the distal intramural portion or the isthmus of the fallopian tubes 100. The dye-releasing agent 50 preferably includes a biodegradable matrix or shell along with the dye. For example, in one aspect the dye-releasing agent 50 is formed by microspheres having a dye compound or material contained therein. The dye-releasing agent 50 may also be embedded within a polymer or gel material that slowly degrades over time. Upon degradation of the matrix or shell, the dye is released on the back side (e.g., distally toward peritoneal cavity) of the site of occlusion 104.

Still referring to FIG. 4, the elongate member 12 with the interrogation element 20 can then be advanced into the uterine cavity 102. The interrogation element 20, which may include a camera or other imaging modality, can then be used to inspect the occlusion site 104 for the presence or absence of any dye. The presence of any dye would indicate a lack of total occlusion as the dye has leaked across the occlusion site 104. The dye-releasing agent 50 may be tailored to dissolve or degrade over a pre-set period of time corresponding to the anticipated time that full or total occlusion of the fallopian tubes 100 should be achieved. For example, the dye-releasing agent 50 may be designed to degrade several weeks after implantation such that occlusion site is tested at a time period when normal scarring processes by the body (i.e., fibrosis) would completely occlude the fallopian tube 100. While the elongate member 12 with the interrogation element 20 may be used to inspect for the presence of dye it is also possible that other imaging tools such as a hysteroscope may be used by the physician to check for dye.

While embodiments of the present invention have been shown and described, various modifications may be made without departing from the scope of the present invention. The invention, therefore, should not be limited, except to the following claims, and their equivalents.

What is claimed is:

1. A device for verifying occlusion of the fallopian tube in a female subject comprising:
an elongate member dimensioned to be slidably disposed within the uterine cavity of the subject; and
an ultrasonic transducer positioned at a distal end of the elongate member.

2. The device of claim 1, wherein the elongate member has a proximal handle.

3. The device of claim 1, further comprising a monitor for displaying images obtained from the ultrasonic transducer.

4. The device of claim 1, wherein the elongate member is dimensioned to be slidably disposed within a lumen of a separate occlusion catheter.

5. The device of claim 1, wherein the elongate member includes an occlusion element disposed on a distal end thereof.

6. The device of claim 5, wherein the occlusion element comprises an RF electrode.

7. The device of claim 1, wherein the elongate member is steerable.

8. The device of claim 1, wherein the elongate member is pre-formed.

9. A device for verifying occlusion of the fallopian tube in a female subject comprising:
an elongate member dimensioned to be slidably disposed within the uterine cavity of the subject; and
an impedance measuring electrode positioned at a distal end of the elongate member.

10. The device of claim 9, further comprising an electrical current source coupled the impedance measuring electrode.

11. The device of claim 9, wherein the elongate member has a proximal handle.

12. The device of claim 9, wherein the elongate member is dimensioned to be slidably disposed within a lumen of a separate occlusion catheter.

13. The device of claim 12, wherein occlusion catheter includes an RF ablation electrode disposed at a distal end thereof.

14. The device of claim 13, wherein the elongate member includes an occlusion element disposed on a distal end thereof.

15. The device of claim 14, wherein the occlusion element comprises an RF electrode.

16. The device of claim 9, wherein the elongate member is steerable.

17. The device of claim 1, wherein the elongate member is pre-formed.

18. A device for verifying occlusion of the fallopian tube in a female subject comprising:
an elongate member dimensioned to be slidably disposed within the uterine cavity of the subject;
a dye applicator disposed on a distal end of the elongate member; and
a visualization tool disposed on a distal portion of the elongate member.

19. The device of claim 18, wherein the dye applicator is selected from the group consisting of a sponge, brush, and dye ejection port.

20. The device of claim 18, wherein the visualization tool comprises a camera.

21. The device of claim 18, wherein the visualization tool comprise a magnetic resonance imaging element.

22. A device for verifying occlusion of the fallopian tube in a female subject comprising:
an elongate member dimensioned to be slidably disposed within the uterine cavity of the subject;
a magnetic resonance imaging element disposed on a distal portion of the elongate member.

23. The device of claim 22, wherein the elongate member has a proximal handle.

24. The device of claim 22, further comprising a monitor for displaying images obtained from the magnetic resonance imaging element.

25. The device of claim 22, wherein the elongate member is dimensioned to be slidably disposed within a lumen of a separate occlusion catheter.

26. The device of claim 22 wherein the elongate member includes an occlusion element disposed on a distal end thereof.

27. The device of claim 26, wherein the occlusion element comprises an RF electrode.

28. The device of claim 22, wherein the elongate member is steerable.

29. The device of claim 22, wherein the elongate member is pre-formed.

30. A method for verifying occlusion of the fallopian tube in a female subject comprising:
placing a dye-releasing agent within the fallopian tube at a location that is distal of a putative occlusion location;
occluding the fallopian tube;
inserting a visualization tool into the uterus and visualizing the occlusion for the presence or absence of dye.

31. The method of claim 30, wherein the dye-releasing agent is biodegradable.

32. The method of claim 30, wherein the dye-releasing agent comprises microspheres having dye contained therein.

33. The method of claim 30, wherein the imaging tool comprises a hysteroscope.
